(19) United States
(12) Patent Application Publication
(10) Pub. No.: US 2017/0081192 A1
(43) Pub. Date: Mar. 23, 2017

(54) ORTHO-TERPHENYLS FOR THE PREPARATION OF GRAPHENE NANORIBBONS

(71) Applicants: BASF SE, Ludwigshafen (DE); Max-Planck-Gesellschaft zur Foerderung der Wissenschaften e.V., Muenchen (DE)

(72) Inventors: Matthias Georg SCHWAB, Mannheim (DE); Klaus MUELLEN, Koeln (DE); Xinliang FENG, Dresden (DE); Tim DUMSLAFF, Koblenz (DE); Pascal RUFFIEUX, Plasselb (DE); Roman FASEL, Zuerich (CH)

(73) Assignees: BASF SE, Ludwigshafen (DE); Max-Planck-Gesellschaft zur Foerderung der Wissenschaften e.V., Muenchen (DE)

(21) Appl. No.: 15/311,418

(22) PCT Filed: May 12, 2015
(86) PCT No.: PCT/EP2015/060421

§ 371 Date: Nov. 15, 2016

(30) Foreign Application Priority Data
May 15, 2014 (EP) 14168466.2

(51) Int. Cl. C01B 31/04; C08G 61/10; H01L 51/00

(52) U.S. Cl. CPC C01B 31/0446; C08G 61/10; H01L 51/0045; C01B 2204/06; C01B 2204/22; B82Y 40/00

(57) ABSTRACT

The present invention concerns ortho-Terphenyls of general formula (I); wherein R¹, R², R³ and R⁴ are independently selected from the group consisting of H; CN; NO₂; and saturated, unsaturated or aromatic C₁-C₄₀ hydrocarbon residues, which can be substituted 1- to 5-fold with F, CI, OH, NH₂, CN and/or NO₂, and wherein one or more —CH₂-groups can be replaced by —O—, —NH—, —S—, —C(═O)O—, —OC(═O)— and/or —C(═O)—; and X and Y are the same or different, and selected from the group consisting of F, CI, Br, I, and OTf (trifluoromethanesulfonate); and their use for the preparation of graphene nanoribbons as well as a process for the preparation of graphene nanoribbons from said ortho-Terphenyls.

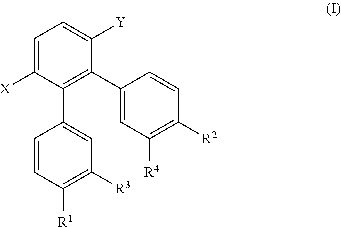

The present invention concerns ortho-terphenyls and their use for the preparation of graphene nanoribbons as well as a process for the preparation of graphene nanoribbons from said ortho-terphenyls.

Graphene consists of two-dimensional carbon layers and possesses a number of outstanding properties. It is not only harder than diamond, extremely tear-resistant and impermeable to gases, but it is also an excellent electrical and thermal conductor. Due to these outstanding properties, graphene has received considerable interest in physics, material science and chemistry. Transistors on the basis of graphene are considered to be potential successors for the silicon components currently in use. However, as graphene is a semi-metal it lacks, in contrast to silicon, an electronic band gap and therefore has no switching capability which is essential for electronic applications.

Graphene nanoribbons (often abbreviated GNRs) are strips of graphene with ultra-thin width that are derived from graphene lattice. They are promising building blocks for novel graphene based electronic devices. Beyond the most important distinction between electrically conducting zig-zag edge (ZGNR) and predominantly semiconducting armchair edge graphene nanoribbons (AGNRs), more general variations of the geometry of a GNR allow for gap tuning through one-dimensional (ID) quantum confinement. In general, increasing the ribbon width leads to an overall decrease of the band gap, with superimposed oscillation features that are maximized for AGNRs.

Standard ‘top-down’ methods for the preparation of GNRs, such as the lithographical patterning of graphene lattices and the unzipping of carbon nanotubes (e.g. described in US 2010/0047154 and US 2011/0097258), give only mixtures of different GNRs. In addition, the proportion of nanoribbons having widths below 10 nm is quite low or even zero. However, for high-efficiency electronic devices, the width of the graphene nanoribbons needs to be precisely controlled and is preferably below 10 nm, and their edges need to be smooth because even minute deviations from the ideal edge shape seriously degrades the electronic properties.

Due to the inherent limitations of such ‘top-down’ methods the realization of structurally well-defined GNRs has remained elusive. ‘Bottom-up’ chemical synthetic approaches through solution-mediated cyclodehydrogenation reactions (e.g. J. Wu, L. Gherghel, D. Watson, J. Li, Z. Wang, C. D. Simpson, U. Kolb, K. Mullen, Macromolecules 2003, 36, 7082-7089; L. Dossel, L. Gherghel, X. Feng, K. Mullen, Angew. Chem. Int. Ed. 2011, 50, 2540-2543; Y. Fogel, L. Zhi, A. Rouhanipour, D. Andrienko, H. J. Rader, K. Mullen, Macromolecules 2009, 42, 6878-6884; and A. Narita et al., Nature Chemistry 2014, 6, 126-132) and surface-assisted cyclodehydrogenation reactions (e.g. J. Cai et al., Nature 2010, 470-473; S. Blankenburg et al., ACS Nano 2012, 6, 2020; S. Linden et al., Phys. Rev. Lett. 2012, 108, 216801) have recently emerged as promising routes for synthesizing GNRs.

In contrast to ‘top-down’ methods, the ‘bottom-up’ chemical synthetic approaches based on solution-mediated or surface-assisted cyclodehydrogenation reactions offer the opportunity to make well-defined and homogeneous GNRs by reacting tailor-made three dimensional polyphenylene precursors. These polyphenylene-based polymeric precursors are built up from small molecules whose structure can be tailored within the capabilities of modern synthetic chemistry.

However, all these ‘bottom-up’ approaches have so far only allowed the preparation of minute amounts of graphene nanoribbons. Moreover, the graphene nanoribbons obtained are frequently ill-defined due to statistically arranged “kinks” in their backbone, or have only low molecular weights.

It is thus an object of the present invention to provide new processes for the preparation of graphene nanoribbons. It is a further object of the present invention to provide suitable oligophenylene monomers and suitable polymeric precursors for the preparation of graphene nanoribbons.

The problem is solved by an ortho-terphenyl of general formula (I);

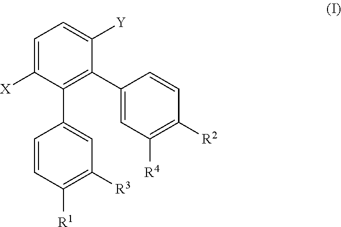

wherein
R¹, R², R³ and R⁴ are independently selected from the group consisting of H; CN; NO₂; and saturated, unsaturated or aromatic C₁-C₄₀ hydrocarbon residues, which can be substituted 1- to 5-fold with F, Cl, OH, NH₂, CN and/or NO₂, and wherein one or more —CH₂-groups can be replaced by —O—, —NH—, —S—, —C(═O)O—, —OC(═O)— and/or —C(═O)—; and
X and Y are the same or different, and selected from the group consisting of F, Cl, Br, I, OTf (trifluoromethanesulfonate).

Preferably, R¹, R², R³ and R⁴ are independently selected from the group consisting of H, unsubstituted C₁-C₄₀ alkyl residues, and unsubstituted C₁-C₄₀ alkoxy residues.

More preferred, R¹ and R² are independently selected from the group consisting of H, unsubstituted C₁-C₂₀ alkyl residues, and unsubstituted C₁-C₂₀ alkoxy residues; and R³ and R⁴ are H. In one embodiment of the present application, R¹ and R² are H.

In the context of the present invention, the expression “C₁-C₄₀ hydrocarbon residues” includes all kind of residues consisting of carbon and hydrogen atoms. Examples are linear or branched C₁-C₄₀ alkyl, linear or branched C₂-C₄₀ alkenyl, linear or branched C₂-C₄₀ alkynyl, and C₆-C₄₀ aryl.

C₁-C₄₀ alkyl residues can be linear or branched, where possible. Examples are methyl, ethyl, n-propyl, isopropyl, n-butyl, sec.-butyl, isobutyl, tert.-butyl, n-pentyl, 2-pentyl, 3-pentyl, 2,2-dimethylpropyl, 1,1,3,3-tetramethylpentyl, n-hexyl, 1-methylhexyl, 1,1,3,3,5,5-hexamethylhexyl, n-heptyl, isoheptyl, 1,1,3,3-tetramethylbutyl, 1-methylheptyl, 3-methylheptyl, n-octyl, 1,1,3,3-tetramethylbutyl and 2-ethylhexyl, nonyl, decyl, undecyl, dodecyl, tridecyl, tetradecyl, pentadecyl, hexadecyl, heptadecyl, octadecyl, nonadecyl, eicosanyl, heneicosanyl, docosanyl, tricosanyl, tetracosanyl, pentacosanyl, hexacosanyl, heptacosanyl, octacosanyl, nonacosanyl, triacontanyl, hentriacontanyl, dotriacontanyl, tritriacontanyl, tetratriacontanyl, pentatriacontanyl, hexatriacontanyl, heptatriacontanyl, octatriacontanyl, nonatriacontanyl, and tetracontanyl.

C₂-C₄₀ alkenyl residues are straight-chain or branched alkenyl residues, e.g. vinyl, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl and n-octadec-4-enyl.

C₂-C₄₀ alkynyl residues are straight-chain or branched. Examples are, ethynyl, 1-propyn-3-yl, 1-butyn-4-yl, 1-pentyn-5-yl, 2-methyl-3-butyn-2-yl, 1,4-pentadiyn-3-yl, 1,3-pentadiyn-5-yl, 1-hexyn-6-yl, cis-3-methyl-2-penten-4-yn-1-yl, trans-3-methyl-2-penten-4-yn-1-yl, 1,3-hexadiyn-5-yl, 1-octyn-8-yl, 1-nonyn-9-yl, 1-decyn-10-yl, and 1-tetracosyn-24-yl.

Examples for C₆-C₄₀ aryl residues are phenyl, naphthyl, biphenylyl, terphenylyl, pyrenyl, fluorenyl, phenanthryl, anthryl, tetracyl, pentacyl or hexacyl.

C₁-C₄₀ alkoxy groups are straight-chain or branched alkoxy groups, e.g. methoxy, ethoxy, n-propoxy, isopropoxy, n-butoxy, sec-butoxy, tert-butoxy, amyloxy, isoamyloxy, tert-amyloxy, heptyloxy, octyloxy, isooctyloxy, nonyloxy, decyloxy, undecyloxy, dodecyloxy, tetradecyloxy, pentadecyloxy, hexadecyloxy, heptadecyloxy, octadecyloxy, nonadecyloxy, eicosanyloxy, heneicosanyloxy, docosanyloxy, tricosanyloxy, tetracosanyloxy, pentacosanyloxy, hexacosanyloxy, heptacosanyloxy, octacosanyloxy, nonacosanyloxy, triacontanyloxy, hentriacontanyloxy, dotriacontanyloxy, tritriacontanyloxy, tetratriacontanyloxy, pentatriacontanyloxy, hexatriacontanyloxy, heptatriacontanyloxy, octatriacontanyloxy, nonatriacontanyloxy, and tetracontanyloxy.

The problem of the present invention is further solved by the use of the ortho-terphenyl of general formula (I), for the preparation of graphene nanoribbons.

Another aspect of the present invention is therefore a process for the preparation of graphene nanoribbons comprising the steps of
(a) polymerizing the ortho-terphenyl of general formula (I) to form a polymeric precursor having repeating units of general formula (II),

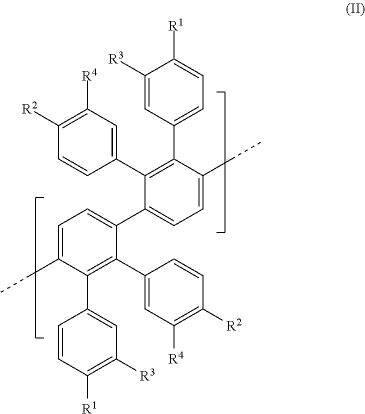

wherein R¹, R², R³ and R⁴ are as defined above; and
(b) cyclodehydrogenating the polymeric precursor to form graphene nanoribbons having repeating units of general formula (III),

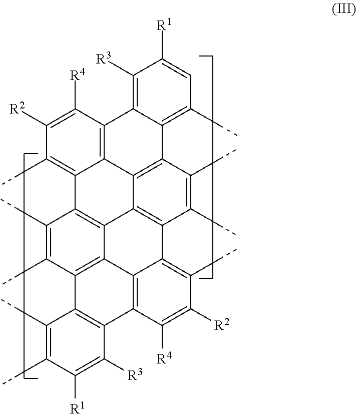

wherein R¹, R², R³ and R⁴ are as defined above.

In a preferred embodiment of the present invention, (a) the polymerization is performed in solution. For example, the polymeric precursor having repeating units of general formula (II) can be obtained by Yamamoto-polycondensation (T. Yamamoto, Progr. Polym. Sci. 1992, 17, 1153-1205; T. Yamamoto, Bull. Chem. Soc. Jpn. 1999, 72, 621-638; T. Yamamoto, T. Kohara, A. Yamamoto, Bull. Chem. Soc. Jpn. 1981, 54, 1720-1726.) in dimethylformamide (DMF) or in a mixture of toluene and DMF. Suitable catalysts for Yamamoto-polycondensation can be prepared from a stoichiometric mixture of bis(cyclooctadiene)nickel(0), 1,5-cyclooctadiene and 2,2′-bipyridine e.g. in a mixture of toluene and DMF. Depending on the particular substituents R¹ and R², the polycondensation reaction is carried out at temperatures of from 50 to 110° C., preferably at temperatures of from 70 to 90° C. The quenching of the Yamamoto-polycondensation reaction and the decomposition of nickel residues is achieved by carefully dropping the reaction mixture into dilute methanolic hydrochloric acid. Usually, a white precipitate is being formed which can be collected by filtration. Further suitable polycondensation reactions rely, for example, on Ullmann-type couplings and Glaser-type couplings. With a suitable co-monomer, the ortho-terphenyl can also be applied for example to Suzuki-Miyaura-type couplings, Negishi-type couplings, Stille-type couplings and Kumada-type couplings.

In one embodiment of the present invention, the (b) cyclodehydrogenation is performed in solution. For example, the preparation of the graphene nanoribbons having repeating units of general formula (III) can be performed using Lewis acids like ferric chloride (FeCl₃), molybdenum chloride (MoCl₅) or copper triflate (Cu(OTf)₂) in a mixture of dichloromethane and nitromethane. Alternatively, the preparation of graphene nanoribbons can be carried out using phenyliodine(III) bis(trifluoroacetate) (PIFA) and BF₃ etherate in anhydrous dichloromethane. It is known that PIFA when activated by a Lewis acid readily reacts with a broad range of substrates to give biaryl products in excellent yields (Takada, T.; Arisawa, M.; Gyoten, M.; Hamada, R.; Tohma, H.; Kita, Y. J. Org. Chem. 1998, 63, 7698-7706). Furthermore, it can be applied to the synthesis of triphenylenes (King, B. T.; Kroulik, J.; Robertson, C. R.; Rempala, P.; Hilton, C. L.; Korinek, J. D.; Gortari, L. M. J. Org. Chem. 2007, 72, 2279-2288.) and hexa-peri-hexabenzocoronene (HBC) derivatives (Rempala, P.; Kroulík, J.; King, B. T. J. Org. Chem. 2006, 71, 5067-5081.). Importantly, undesired chlorination, which is frequently observed when applying ferric chloride, is ruled out by this procedure. Suitable variations of such types of cyclodehydrogenation reactions can be found in the article “Cyclodehydrogenation in the Synthesis of Graphene-Type Molecules” (M. Kivala, D. Wu, X. Feng, C. Li, K. Mullen, Materials Science and Technology 2013, 373-420), and the literature cited therein.

In general, the molecular weight of the graphene nanoribbons obtained by cyclodehydrogenation performed in solution varies from 1,000 to 1,000,000 g/mol, preferably from 20,000 to 200,000 g/mol.

In another preferred embodiment of the present invention, (a) the polymerization and (b) the cyclodehydrogenation are performed on inert surfaces. Accordingly, the graphene nanoribbons having repeating units of general formula (III) are prepared by direct growth on this surfaces under high vacuum conditions. Thereby, the ortho-terphenyl of general formula (I) is firstly polymerized at elevated temperatures to form the polymeric precursor having repeating units of general formula (II), which is then at further elevated temperatures reacted to form graphene nanoribbons having repeating units of general formula (III).

Surface-assisted bottom-up approaches using ultra-high vacuum (UHV) conditions have been described in J. Cai et al., Nature 466, pp. 470-473 (2010) and in a small number of publications since then (S. Blankenburg et al., ACS Nano 2012, 6, 2020; S. Linden et al., Phys. Rev. Lett. 2012, 108, 216801). Alternatively, the surface-assisted bottom-up approach disclosed in WO 2014/045148 A1 can be used. This approach has the advantage that no ultra-high vacuum needs to be applied.

In the context of the present invention, the expression “inert surfaces” includes surfaces of all kinds of solid substrates enabling the adsorption/deposition of the ortho-terphenyl of general formula (I) and/or or the polymeric precursor having repeating units of general formula (II), and the subsequent polymerization and/or cyclodehydrogenation, without reacting irreversibly with said compounds themselves. The “inert surface” may preferably be acting as a catalyst for the polymerization and/or cyclodehydrogenation reaction. The inert surface can be a metal surface such as a Au, Ag, Cu, Al, W, Ni, Pt, or a Pd surface, preferably a Au and/or Ag surface. The surface may also be a metal oxide surface such as silicon oxide, silicon oxynitride, hafnium silicate, nitrided hafnium silicates, zirconium silicate, hafnium dioxide and zirconium dioxide, or aluminum oxide, copper oxide, iron oxide. The surface may also be made of a semiconducting material such as silicon, germanium, gallium arsenide, silicon carbide, and molybdenum disulfide. The surface may also be a material such as boron nitride, sodium chloride, or calcite. The surface may be electrically conducting, semiconducting, or insulating.

The deposition on the surface may be done by a vacuum deposition (sublimation) process, a solution based process such as spin coating, spray coating, dip coating, printing, electrospray deposition, or a laser induced desorption or transfer process. The deposition process may also be a direct surface to surface transfer. Preferably the deposition is done by a vacuum deposition process. Preferably it is a vacuum sublimation process.

Depending on the surface-assisted approach discussed above, the pressures applied in the reaction steps (a) and (b) are usually below 10⁻⁵ mbar, frequently below 10⁻⁵ mbar.

Preferably, the polymerization in step (a) is induced by thermal activation. However, any other energy input which induces polymerization such as, for example, radiation can be used as well. The activation temperature is dependent on the employed surface and the substitution pattern of the ortho-terphenyl of general formula (I). Usually, the temperature is in the range of from 100 to 300° C.

Optionally, step (a) can be repeated one or several times before carrying out partial or complete cyclodehydrogenation in step (b).

As indicated above, step (b) of the process of the present invention includes at least partially, preferably completely cyclodehydrogenating the polymeric precursor having repeating units of general formula (II) to form the graphene nanoribbons having repeating units of general formula (III). The cyclodehydrogenation reaction is usually performed at temperatures in the range of from 200 to 500° C.

Preferably, the surface-assisted approach does not comprise any intermediate steps in between the process steps (a) and (b). Steps (a) and (b) can directly follow each other and/or overlap.

In general, the molecular weight of the graphene nanoribbons having repeating units of general formula (III) obtained by direct growth on surfaces varies from 2,000 to 1,000,000 g/mol, preferably from 4,000 to 100,000 g/mol.

Covalently bonded two-dimensional molecular arrays can be efficiently studied by scanning tunneling microscope (STM) techniques. Examples of surface-confined covalent bond formation involve Ullmann coupling, imidization, crosslinking of porphyrins and oligomerization of heterocyclic carbenes and polyamines. A chemistry-driven protocol for the direct growth of graphene nanoribbons and graphene networks on surfaces has been very recently established by the groups of Müllen (MPI-P Mainz, Germany) and Fasel (EMPA Dübendorf, Switzerland) (Bieri, M.; Treier, M.; Cai, J.; Aït-Mansour, K.; Ruffieux, P.; Gröning, O., Gröning, P.; Kastler, M.; Rieger, R.; Feng, X.; Müllen, K.; Fasel, R.; Chem. Commun. 2009, 45, 6919; Bieri, M.; Nguyen, M. T.; Gröning, O.; Cai, J.; Treier, M.; Aït-Mansour, K.; Ruffieux, P.; Pignedoli, C. A.; Passerone, D.; Kastler, M.; Müllen, K.; Fasel, R.; J. Am. Chem. Soc. 2010, 132, 16669; Treier, M.; Pignedoli, C. A.; Laino, T.; Rieger, R.; Müllen, K.; Passerone, D.; Fasel, R. Nature Chemistry 2011, 3, 61; Cai, J.; Ruffieux, P.; Jaafar, R.; Bieri, M.; Braun, T.; Blankenburg, S.; Muoth, M.; Seitsonen, A. P.; Saleh, M.; Feng, X.; Müllen, K.; Fasel, R. Nature 2010, 466, 470-473.). Without being bound by theory it can be concluded from these studies that the nanoribbon formation on the metal surface proceeds via a radical pathway. After deposition of the functionalized monomer on the surface via ultra high vacuum (UHV) sublimation (10⁻¹¹ to 10⁻⁵ mbar, preferably 10⁻¹⁰ to 10⁻⁷ mbar), dehalogenation is believed to occur upon thermal activation by annealing to 100 to 200° C. This generates biradical species that diffuse on the surface and couple to each other resulting in the formation of carbon-carbon bonds. These radical addition reactions proceed at intermediate thermal levels (100 to 300° C., preferably 150 to 220° C.) and are the prerequisite for the subsequent cyclodehydrogenation at higher temperatures (200 to 500° C., preferably 380 to 420° C.). Only if polymeric species of sufficient molecular weight are formed during the first stage, the full graphitization of the molecules will proceed subsequently with the thermal desorption of the material from the surface being avoided.

For UHV surface-assisted polymerization and cyclodehydrogenation, functional monomers of sufficiently high rigidity and planarity are needed which assist in the flat orientation on the metal substrate. Also, the method allows for the topological tailoring of the graphene nanoribbons as their shape is determined by the functionality pattern and geometry of the precursor monomers. Solubilizing alkyl chains are not needed in the monomer design as no solvent-based process is involved in this surface-bound protocol.

A further aspect of the present application is a polymeric precursor for the preparation of graphene nanoribbons, having repeating units of general formula (II),

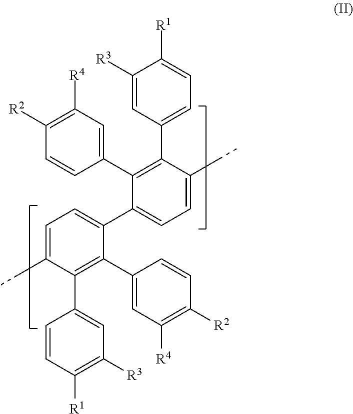

wherein R¹, R², R³ and R⁴ are as defined above.

Another aspect of the present application are the graphene nanoribbons having repeating units of general formula (III),

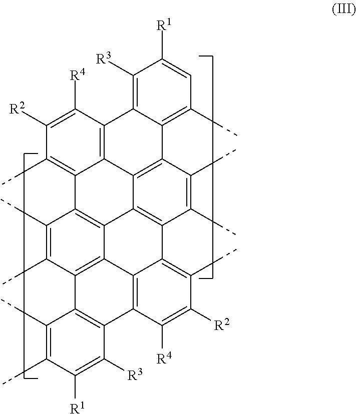

wherein R¹, R², R³ and R⁴ are as defined above.

The ortho-terphenyl of general formula (I) can be synthesized according to Schemes 1 to 3 shown below. Reaction conditions and solvents used are purely illustrative; of course other conditions and solvents can also be used and can easily be determined by the person skilled in the art. As starting material for the synthesis of the ortho-terphenyl of general formula (I), the commercially available 2,5-dihaloaniline 1 is used (Scheme 1). In the first step of the reaction sequence, 2,5-dihaloaniline 1 is reacted with chloralhydrate 2 and hydroxylamine hydrochloride under basic conditions to form (2,5-dihalophenyl)-2-(hydroxyimino)acetamide 3. Then, the (2,5-dihalophenyl)-2-(hydroxyimino)acetamide 3 is subjected to sulfuric acid at elevated temperatues to yield 4,7-dihaloindoline-2,3-dione 4.

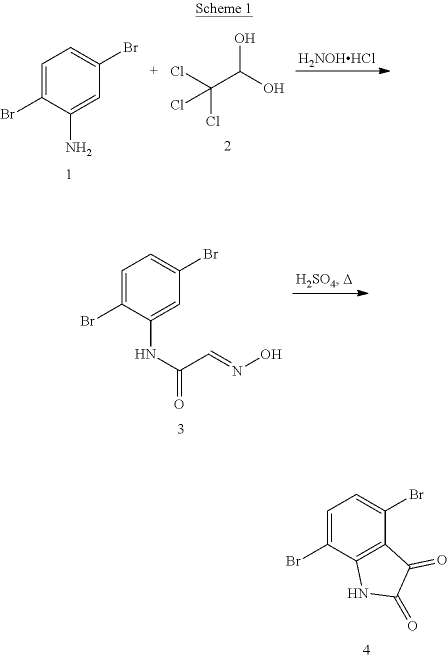

To a solution of 4,7-dihaloindoline-2,3-dione 4 and sodium hydroxide in water is added an aqueous solution of hydrogen peroxide, and the reaction mixture is heated to 50° C. (Scheme 2). After cooling and acidic work-up, the 2-amino-3,6-dihalobenzoic acid 5 is obtained, which is subsequently reacted with iodine and isoamylnitrite to yield 1,4-dibromo-2,3-diiodobenzene 6.

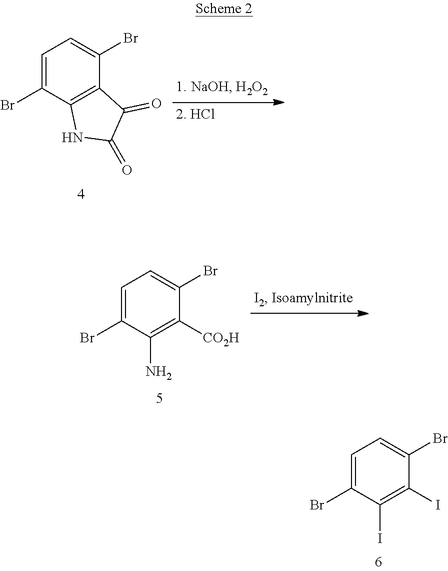 [SMILES: O=C1Nc2c(Br)ccc(Br)c2C1=O ; Nc1c(Br)ccc(Br)c1C(=O)O ; Brc1ccc(Br)c(I)c1I]

Then, 1,4-dibromo-2,3-diiodobenzene 6 is subjected to two consecutive Suzuki coupling reactions (Scheme 3). The first Suzuki coupling reaction of 1,4-dibromo-2,3-diiodobenzene 6 with boronic acid 9 can e.g. be performed at elevated temperatures in dioxane in the presence of catalytic amounts of tetrakis(triphenylphosphine)palladium(0) (Pd(PPh₃)₄) and a base like, for example, sodium carbonate. The so obtained monocoupled biphenyl (IV) can be subjected to the second Suzuki reaction. The ortho-terphenyl of general formula (I) can e.g. be synthesized by heating a reaction mixture of the monocoupled biphenyl (IV), arylbronic acid 10, a palladium(0) catalyst and a base in dioxane to 100° C. for several days. After purification, the ortho-terphenyl of general formula (I) can be subjected to the polymerization.

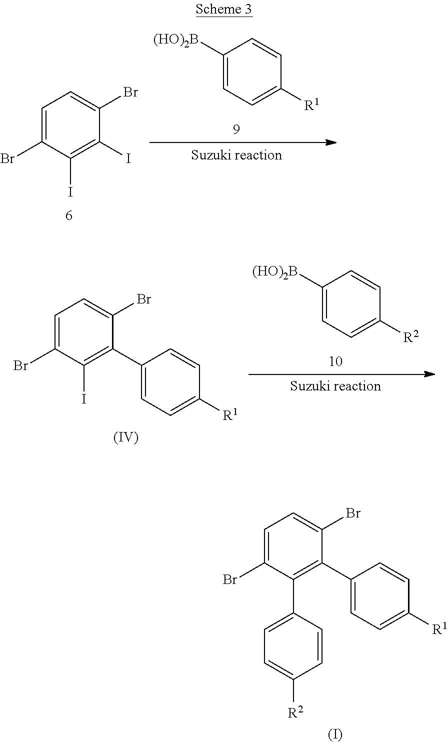

Various articles of manufacture including electronic devices, optical devices, and optoelectronic devices, such as field effect transistors (e.g. thin film transistors), photovoltaics, organic light emitting diodes (OLEDs), complementary metal oxide semiconductors (CMOSs), complementary inverters, D flip-flops, rectifiers, and ring oscillators, that make use of the graphene nanoribbons disclosed herein also are within the scope of the present invention as are methods of making the same.

Another aspect of the present invention is therefore the use of the graphene nanoribbons, having repeating units of general formula (III) as defined above, in an electronic, optical, or optoelectronic device. Preferably, the device is an organic field effect transistor device, an organic photovoltaic device, or an organic light-emitting diode.

The present invention, therefore, further provides methods of preparing a semiconductor material exhibiting a well-defined electronic band gap that can be tailored to specific applications by the choice of molecular precursor. The methods can include preparing a composition that includes one or more of the compounds of the invention disclosed herein dissolved or dispersed in a liquid medium such as a solvent or a mixture of solvents, depositing the composition on a substrate to provide a semiconductor material precursor, and processing (e.g. heating) the semiconductor precursor to provide a semiconductor material (e.g. a thin film semiconductor) that includes one or more of the compounds disclosed herein. In various embodiments, the liquid medium can be an organic solvent, an inorganic solvent such as water, or combinations thereof. In some embodiments, the composition can further include one or more additives independently selected from detergents, dispersants, binding agents, compatibilizing agents, curing agents, initiators, humectants, antifoaming agents, wetting agents, pH modifiers, biocides, and bacteriostats. For example, surfactants and/or polymers (e.g. polystyrene, polyethylene, poly-alphamethylstyrene, polyisobutene, polypropylene, polymethylmethacrylate, and the like) can be included as a dispersant, a binding agent, a compatibilizing agent, and/or an antifoaming agent. In some embodiments, the depositing step can be carried out by printing, including inkjet printing and various contact printing techniques (e.g. screen-printing, gravure printing, offset printing, pad printing, lithographic printing, flexographic printing, and microcontact printing). In other embodiments, the depositing step can be carried out by spin coating, drop-casting, zone casting, dip coating, blade coating, spraying or vacuum filtration.

The present invention further provides articles of manufacture such as the various devices described herein that include a composite having a semiconductor material of the present invention and a substrate component and/or a dielectric component. The substrate component can be selected from doped silicon, an indium tin oxide (ITO), ITO-coated glass, ITO-coated polyimide or other plastics, aluminum or other metals alone or coated on a polymer or other substrate, a doped polythiophene, and the like. The dielectric component can be prepared from inorganic dielectric materials such as various oxides (e.g. SiO₂, Al₂O₃, HfO₂), organic dielectric materials such as various polymeric materials (e.g. polycarbonate, polyester, polystyrene, polyhaloethylene, polyacrylate), and self-assembled superlattice/self-assembled nanodielectric (SAS/SAND) materials (e.g. described in Yoon, M-H. et al., PNAS, 102 (13): 4678-4682 (2005)), as well as hybrid organic/inorganic dielectric materials (e.g. described in US 2007/0181961 A1). The composite also can include one or more electrical contacts. Suitable materials for the source, drain, and gate electrodes include metals (e.g. Au, Al, Ni, Cu), transparent conducting oxides (e.g. ITO, IZO, ZITO, GZO, GIO, GITO), and conducting polymers (e.g. poly(3,4-ethylenedioxythiophene) poly(styrenesulfonate) (PEDOT:PSS), polyaniline (PANI), polypyrrole (PPy). One or more of the composites described herein can be embodied within various organic electronic, optical, and optoelectronic devices such as organic thin film transistors (OTFTs), specifically, organic field effect transistors (OFETs), as well as sensors, capacitors, unipolar circuits, complementary circuits (e.g. inverter circuits), and the like.

A further aspect of the present invention is therefore an electronic, optical, or optoelectronic device comprising a thin film semiconductor, comprising graphene nanoribbons having repeating units of general formula (III) as defined above. Preferably, the device is an organic field effect transistor device, an organic photovoltaic device, or an organic light-emitting diode.

Other articles of manufacture, in which graphene nanoribbons of the present invention are useful, are photovoltaics or solar cells. Compounds of the present invention can exhibit broad optical absorption and/or a very positively shifted reduction potential, making them desirable for such applications. Accordingly, the compounds described herein can be used as n-type semiconductor in a photovoltaic design, which includes an adjacent p-type semiconductor material that forms a p-n junction. The compounds can be in the form of a thin film semiconductor, which can be deposited on a substrate to form a composite. Exploitation of compounds of the present invention in such devices is within the knowledge of a skilled artisan.

Accordingly, another aspect of the present invention relates to methods of fabricating an organic field effect transistor that incorporates a semiconductor material of the present invention. The semiconductor materials of the present invention can be used to fabricate various types of organic field effect transistors including top-gate top-contact capacitor structures, top-gate bottom-contact capacitor structures, bottom-gate top-contact capacitor structures, and bottom-gate bottom-contact capacitor structures.

In certain embodiments, OTFT devices can be fabricated with the present graphene nanoribbons on doped silicon substrates, using SiO₂ as the dielectric, in top-contact geometries. In particular embodiments, the active semiconductor layer which incorporates at least a compound of the present invention can be deposited at room temperature or at an elevated temperature. In other embodiments, the active semiconductor layer which incorporates at least a compound of the present invention can be applied by spin-coating or printing as described herein. For top-contact devices, metallic contacts can be patterned on top of the films using shadow masks, electron beam lithography and lift-off techniques, or other suitable structuring methods that are within the knowledge of a skilled artisan.

The invention is illustrated in more detail by the following examples.

### EXAMPLES

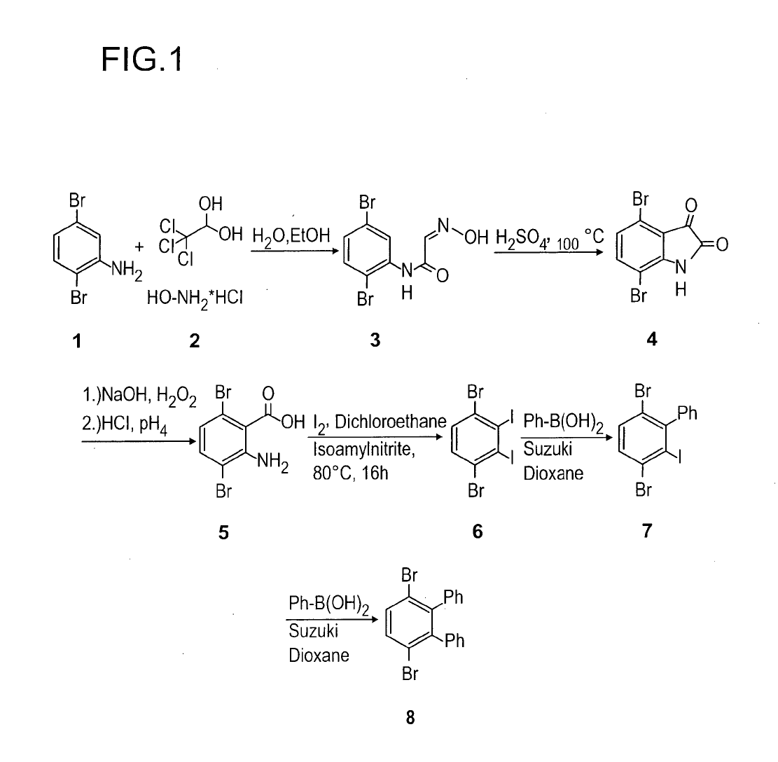

FIGS. 1 to 7 show:

FIG. 1: Synthesis route for 3′,6′-dibromo-1,1′:2′,1″-terphenyl 8 (ortho-terphenyl (I), wherein R¹═R²═R³═R⁴═H, and X═Y═Br).

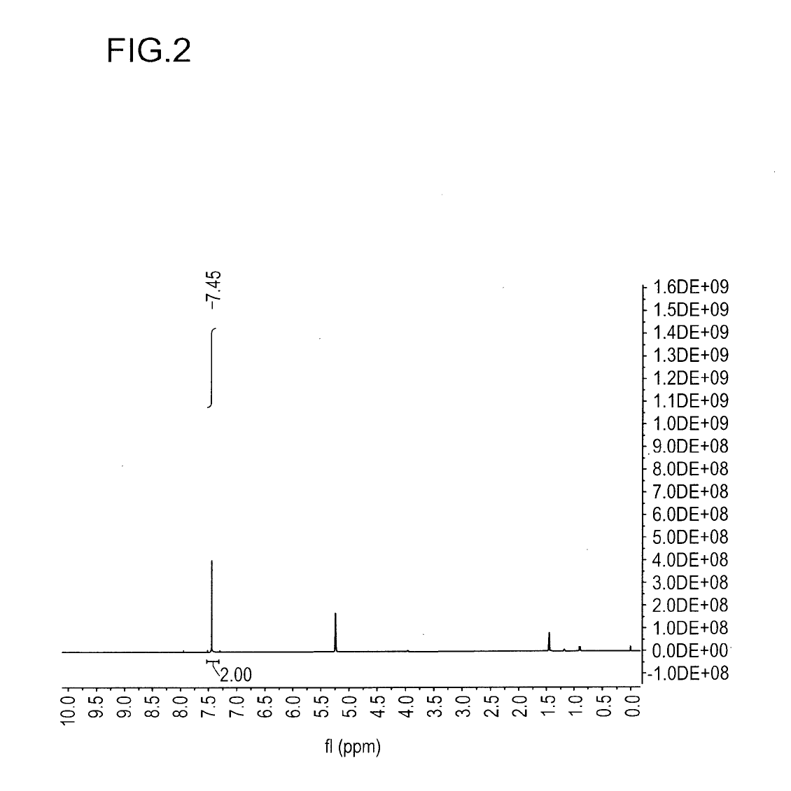

FIG. 2: ¹H NMR (300 MHz, CD₂Cl₂) of 1,4-dibromo-2,3-diiodobenzene 6.

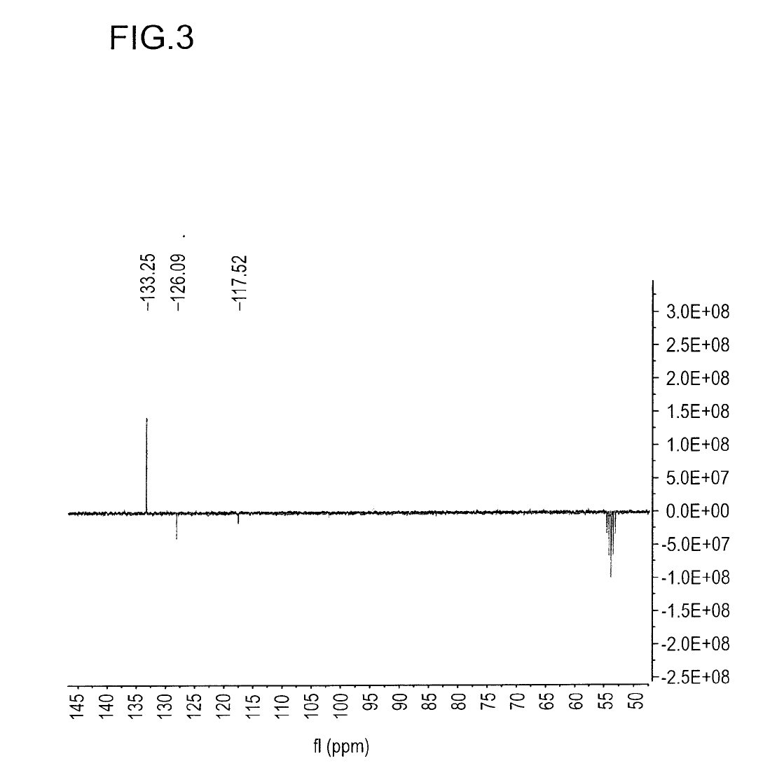

FIG. 3: ¹³C NMR (75 MHz, CD₂Cl₂) of 1,4-dibromo-2,3-diiodobenzene 6.

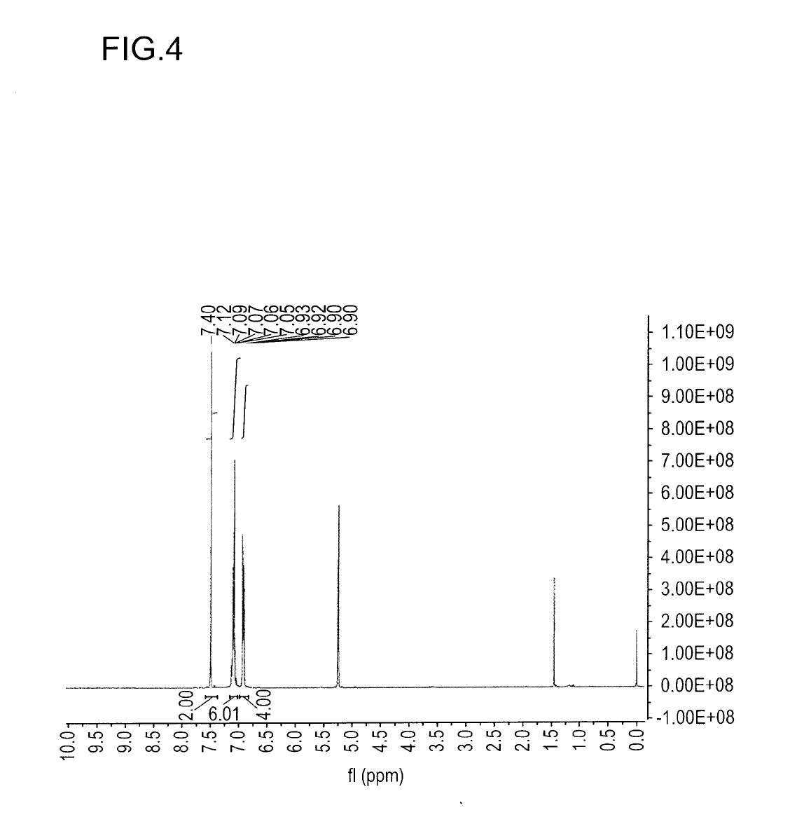

FIG. 4: ¹H NMR (300 MHz, CD₂Cl₂) of 3′,6′-dibromo-1,1′:2′,1″-terphenyl 8.

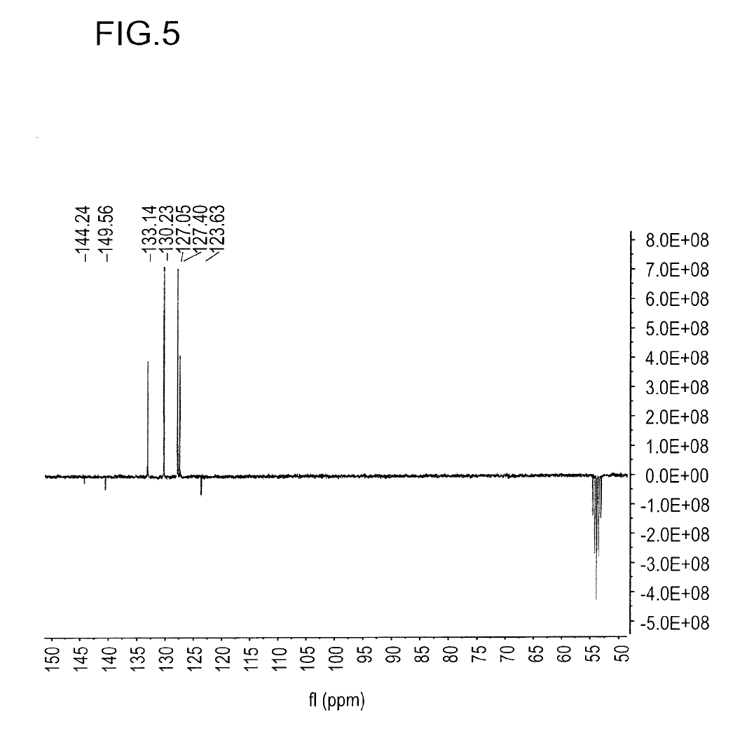

FIG. 5: ¹³C NMR (75 MHz, CD₂Cl₂) of 3′,6′-dibromo-1,1′:2′,1″-terphenyl 8.

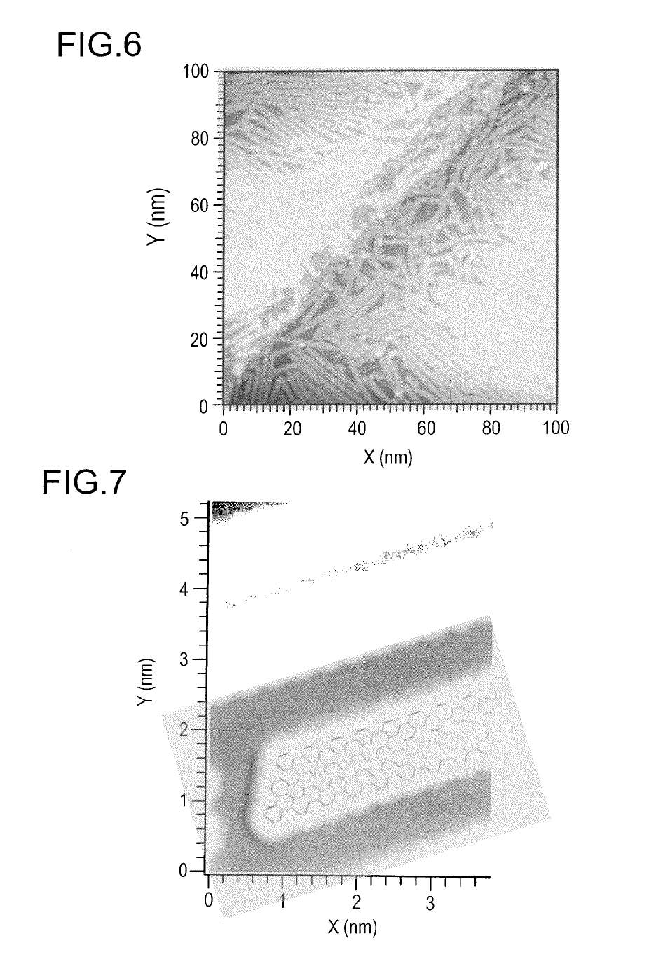

FIG. 6: STM image of the 9-AGNR, obtained from 3′,6′-dibromo-1,1′:2′,1″-terphenyl 8 after polymerization and cyclodehydrogenation on the Au surface.

FIG. 7: Magnification showing the superimposition of the STM image with the chemical model of the AGNR structure.

### EXAMPLE 1

### Preparation of (2,5-dihalophenyl)-2-(hydroxyimino)acetamide 3

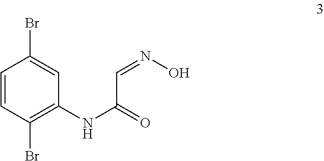 [SMILES: O=C(/C=N\O)Nc1cc(Br)ccc1Br]

(2,5-Dihalophenyl)-2-(hydroxyimino)acetamide 3 was synthesized as described in S.-J. Garden, J.-C. Torres, A.-A. Ferreira, R.-B. Silva, A.-C. Pinto, Tetrahedron Lett. 1997, 38, 1501. Accordingly, in a 1 L round bottomed flask, 10 g (39.85 mmol) 2,5-dihaloaniline 1, 7.91 g (47.82 mmol) chloralhydrate, 4.15 g (59.78 mmol) hydroxylamine hydrochloride and 48 g sodiumsulfate were placed. 300 mL of ethanol and 300 mL of water were added and the reaction mixture was stirred for 12 h at 80° C. After cooling to room temperature, the precipitate was filtered, washed with a mixture of ethylacetate and hexane (1:10) and dried under vacuum to obtain (2,5-dihalophenyl)-2-(hydroxyimino)acetamide 3 as a white solid in 72% yield.

¹H-NMR: (300 MHz, DMSO): δ=12.54 (s, 1H), 9.51 (s, 1H), 8.15 (d, 1H), 7.6 (m, 2H), 7.34 (dd, 1H) ppm.

¹³C-NMR: (300 MHz, DMSO): δ=160.45, 143.10, 136.73, 134.18, 129.15, 126.50, 120.58, 114.96 ppm.

### EXAMPLE 2

### Preparation of 4,7-dihaloindoline-2,3-dione 4

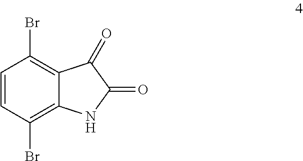 [SMILES: O=C1Nc2c(Br)ccc(Br)c2C1=O]

As described in S.-J. Garden et al., Tetrahedron Lett. 1997, 38, 1501, concentrated sulfuric acid (45 mL) was heated to 50° C. in a 250 mL roundbottom flask. Dried (2,5-dihalophenyl)-2-(hydroxyimino)acetamide 3 (5 g, 15.6 mmol) was added and the reaction mixture heated to 100° C. for 30 min. The resulting purple mixture was cooled to room temperature and poured into ice water (300 mL) to precipitate 4,7-dihaloindoline-2,3-dione 4 as light orange solid. The precipitate was filtered and dried in vacuum to obtain 4 in 56% yield.

¹H-NMR: (300 MHz, DMSO): δ=11.43 (s, 1H), 7.66 (d, 1H), 7.17 (d, 1H) ppm.

¹³C-NMR: (300 MHz, DMSO): δ=181.08, 158.94, 151.06, 140.64, 127.86, 118.36, 103.68 ppm.

### EXAMPLE 3

### Preparation of 2-amino-3,6-dihalobenzoic acid 5

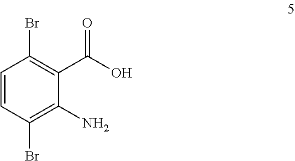 [SMILES: Nc1c(Br)ccc(Br)c1C(=O)O]

2-Amino-3,6-dihalobenzoic acid 5 was synthesized according to a synthesis procedure described in the publication: V. Lisowski, M. Robba, S. Rault, J. Org. Chem. 2000, 65, 4193. Accordingly, 4,7-dihaloindoline-2,3-dione 4 (3 g, 10 mmol) was dissolved in 50 mL 5% sodium hydroxide and heated to 50° C. 30% hydrogen peroxide (50 mL) was added dropwise and the resulting mixture was stirred at 50° C. for an additional 30 min. After cooling to room temperature, the solution was filtered and acidified to pH 4 with 1M hydrochloric acid. The beige precipitate was filtered and dried in vacuum to obtain 2-amino-3,6-dihalobenzoic acid 5 in 65% yield.

¹H-NMR: (300 MHz, DMSO): δ=13.73 (b s, 1H), 7.38 (d, 1H), 6.79 (d, 1H), 5.58 (b s, 1H) ppm.

¹³C-NMR: (300 MHz, DMSO): δ=167.32, 144.12, 134.32, 121.09, 118.96, 107.86 ppm.

### EXAMPLE 4

### Preparation of 1,4-dibromo-2,3-diiodobenzene 6

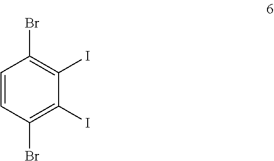

1,4-dibromo-2,3-diiodobenzene 6 was synthesized according to a procedure published in the article: O. S. Miljanic, K. P. C. Vollhardt, G. D. Whitener Synlett 2003, 29-34. To a stirred and refluxed solution of iodine (2.58 g, 10.17 mmol) and isoamyl nitrite (1.64 mL, 12.21 mmol) in 200 mL 1,2-dichloroethane was added dropwise a solution of 2-amino-3,6-dihalobenzoic acid 5 in 15 mL dioxane. The resulting mixture was refluxed for 1 h, cooled to room temperature, filtered and the filtrate washed with 5% aqueous sodium thiosulfate. The organic phase was dried over magnesium sulfate and the solvent evaporated. The resulting residue was purified by flash column chromatography with hexane to obtain 1,4-dibromo-2,3-diiodobenzene 6 in 60% yield as colourless needles. The spectroscopical data is in agreement with the literature values.

¹H-NMR: (300 MHz, CD₂Cl₂): δ=7.45 (s, 2H) ppm.

¹³C-NMR: (300 MHz, CD₂Cl₂): δ=133.25, 128.09, 117.52 ppm.

### EXAMPLE 5

### Preparation of 3′,6′-dibromo-1,1′:2′,1″-terphenyl 8

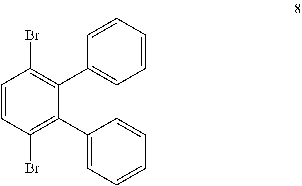 [SMILES: Brc1ccc(Br)c(-c2ccccc2)c1-c1ccccc1]

1,4-dibromo-2,3-diiodobenzene 6 (250 mg, 0.5 mmol) and phenylboronic acid (65.63 mg, 0.5 mmol) were dissolved in 10 mL dioxane and 1 mL of 2 M aqueous sodium carbonate was added. Argon was bubbled through the solution for 45 min and, then, tetrakis(triphenylphosphine)palladium(0) (60 mg, 0.1 mol %) was added. Argon was bubbled through the solution for additional 15 min and the reaction mixture stirred at 80° C. for 2 days. After cooling to room temperature, the solution was extracted with water/dichloromethane, the organic phase dried over magnesium sulfate and the solvent evaporated. The crude mixture was purified by column chromatography (PE:DCM 9:1) to obtain the mono coupled product 7 in 60% yield.

The second iodine was coupled in a similar Suzuki coupling reaction with an additional equivalent of phenylboronic acid. The solution was stirred at 100° C. under Argon for 3 days. The crude reaction mixture was purified by column chromatography (PE:DCM 9:1) to obtain 3′,6′-dibromo-1,1′:2′,1″-terphenyl 8 in 10% yield. The colorless solid can be recrystallized from ethanol.

¹H-NMR: (300 MHz, CD₂Cl₂): δ=7.49 (s, 2H), 7.12-7.05 (m, 6H), 6.93-6.90 (m, 4H) ppm.

¹³C-NMR: (300 MHz, CD₂Cl₂): δ=144.24, 140.56, 133.14, 130.23, 127.85, 127.45, 123.63 ppm.

FD-MS: m/z=388.0

### EXAMPLE 6

### Surface-Assisted Preparation of Graphene Nanoribbons

The Au(111) single crystal (Surface Preparation Laboratory, Netherlands) was used as the substrate for the growth of N=9 armchair graphene nanoribbons (9-AGNR). First the substrate was cleaned by repeated cycles of argon ion bombardment and annealing to 480° C. and then cooled to room temperature for deposition. 3′,6′-dibromo-1,1′:2′,1″-terphenyl 8 was deposited onto the clean surface by sublimation at rates of ˜1 Å/min. Then the Au(111) substrate was post-annealed at 175° C. for 10 min to induce polymerization and at 400° C. for 10 min to form GNRs. A low temperature STM (LT-STM) from Omicron Nanotechnology GmbH, Germany, was used to characterize the morphology of the 9-AGNR samples. The agreement between model and STM image proves that 9-AGNRs can be synthesized from 3′,6′-dibromo-1,1′:2′,1″-terphenyl 8 on Au(111) surfaces (FIG. 6).

1. A process for the preparation of a graphene nanoribbon, the process comprising:
(a) polymerizing an ortho-terphenyl of general formula (I): 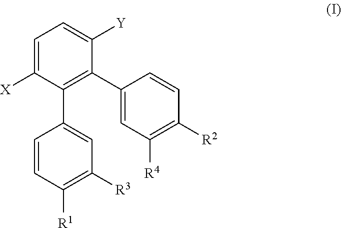
wherein
R¹, R², R³ and R⁴ are each independently selected from the group consisting of H; unsubstituted C₁-C₄₀ alkyl residues; and unsubstituted C₁-C₄₀ alkoxy residues; and
X and Y are each independently selected from the group consisting of F, Cl, Br, I, and OTf
thereby forming a polymeric precursor comprising repeating units of general formula (II) 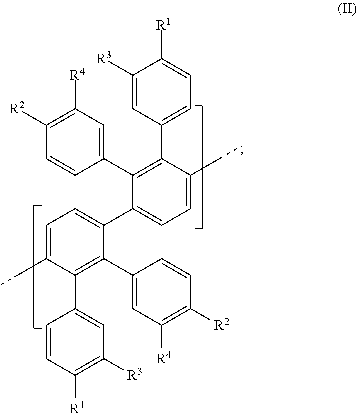
and
(b) cyclodehydrogenating the polymeric precursor of general formula (II) thereby forming a graphene nanoribbon comprising repeating units of general formula (III)

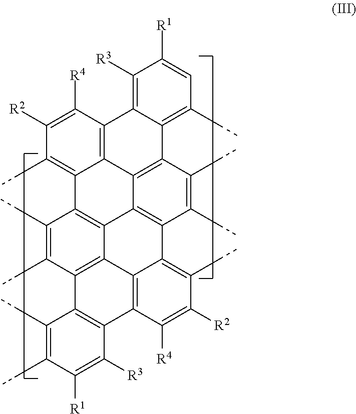

2. The process according to claim 1, wherein R¹ and R² are each independently selected from the group consisting of H, unsubstituted C₁-C₂₀ alkyl residues, and unsubstituted C₁-C₂₀ alkoxy residues; and wherein R³ and R⁴ are H.

3. The process according to claim 1, wherein R¹, R², R³ and R⁴ are H.

4. The process according to claim 1, wherein X and Y are the same.

5. The process according to claim 1, wherein X and Y are Br.

6. (canceled)

7. The process according to claim 1, wherein the olvmerizing (a) is performed in a solution.

8. The process according to claim 1, wherein the cyclodehydrogenating (b) is performed in a solution.

9. The process according to claim 1, wherein the polymerizing (a) and the cyclodehydrogenating (b) are performed on an inert surface.

10. A polymeric precursor for the preparation of a graphene nanoribbon, the polymeric precursor comprising repeating units of general formula (II), 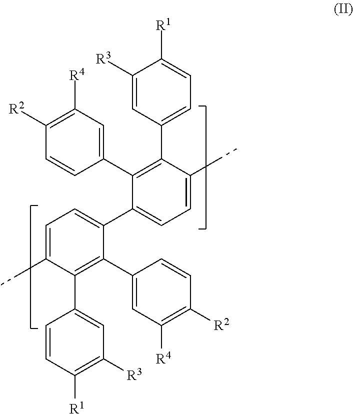
wherein R¹, R², R³ and R⁴ are each independently selected from the group consisting of H; unsubstituted C₁-C₄₀ alkyl residues; and unsubstituted C₁-C₄₀ alkoxy residues.

11. The polymeric precursor according to claim 10, wherein R¹ and R² are each independently selected from the group consisting of H, unsubstituted C₁-C₂₀ alkyl residues, and unsubstituted C₁-C₂₀ alkoxy residues; and wherein R³ and R⁴ are H.

12. The polymeric precursor according to claim 10, wherein R¹, R², R³ and R⁴ are H.

13. A graphene nanoribbon comprising repeating units of general formula (III) 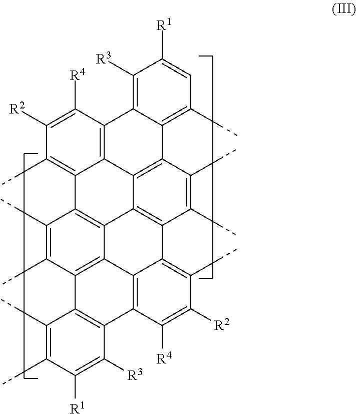
wherein R¹, R², R³ and R⁴ are each independently selected from the group consisting of H; unsubstituted C₁-C₄₀ alkyl residues; and unsubstituted C₁-C₄₀ alkoxy residues
obtained by the process according to claim 1.

14. The graphene nanoribbon according to claim 13, wherein R¹ and R² are each independently selected from the group consisting of H, unsubstituted C₁-C₂₀ alkyl residues, and unsubstituted C₁-C₂₀ alkoxy residues; and wherein R³ and R⁴ are H.

15. The graphene nanoribbon according to claim 14, wherein R¹, R², R³ and R⁴ are H.

16. An electronic, optical, or optoelectronic device comprising the graphene nanoribbon according to claim 13.

17. An electronic, optical or optoelectronic device comprising a thin film semiconductor, the semiconductor comprising the graphene nanoribbon according to claim 13.

18. The electronic, optical or optoclectronic device according to claim 17, wherein the device is at least one selected from the group consisting of an organic field effect transistor device, an organic photovoltaic device, and an organic light-emitting diode.

19. A process for preparing the polymeric precursor comprising repeating units of general formula (II) according to claim 10, the method comprising:
polymerizing an ortho-terphenyl of general formula (I) 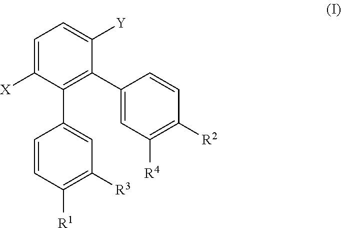
wherein
R¹, R², R³ and R⁴ are each independently selected from the group consisting of H; unsubstituted C₁-C₄₀ alkyl residues; and unsubstituted C₁-C₄₀ alkoxy residues; and
X and Y are each independently selected from the group consisting of F, Cl, Br, I, and OTf
thereby forming the polymeric precursor comprising repeating units of general formula (II).

20. A process for preparing a graphene nanoribbon comprising repeating units of general formula (III), the process comprising:
cyclodehydrogenating the polymeric precursor comprising repeating units of general formula (II) according to claim 10 thereby forming a graphene nanoribbon comprising repeating units of general formula (III) 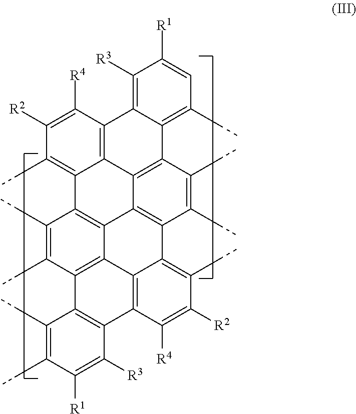
wherein R¹, R², R³ and R⁴ are each independently selected from the group consisting of H; unsubstituted C₁-C₄₀ alkyl residues; and unsubstituted alkoxy residues.
